SEQUENCE LISTING (27 sequences)

SEQ ID NO: 1 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 5 aa, Homo sapiens
GGGGS

SEQ ID NO: 3 - protein, 9 aa, Homo sapiens
GGGGAGGGG

SEQ ID NO: 4 - protein, 9 aa, Homo sapiens
GGGGSGGGG

SEQ ID NO: 5 - protein, 10 aa, Homo sapiens
GGGGGAGGGG

SEQ ID NO: 6 - protein, 19 aa, Homo sapiens
GGGGSGGGGSGGGGSGGGG

SEQ ID NO: 7 - protein, 19 aa, Homo sapiens
GGGGKGGGGKGGGGKGGGG

SEQ ID NO: 8 - protein, 21 aa, Homo sapiens
GGGGGAGGGGAGGGGAGGGGG

SEQ ID NO: 9 - protein, 21 aa, Homo sapiens
GGGGGQGGGGQGGGGQGGGGG

SEQ ID NO: 10 - protein, 21 aa, Homo sapiens
SGGGGQGGGGQGGGGQGGGGG

SEQ ID NO: 11 - protein, 21 aa, Homo sapiens
HGGGGQGGGGQGGGGQGGGGG

SEQ ID NO: 12 - protein, 22 aa, Homo sapiens
PGGGGGQGGGGQGGGGQGGGGG

SEQ ID NO: 13 - protein, 26 aa, Homo sapiens
GGGGGQGGGGQGGGGQGGGGGQGGGG

SEQ ID NO: 14 - protein, 5 aa, Homo sapiens
LRHYY

SEQ ID NO: 15 - protein, 4 aa, Homo sapiens
DIIE

SEQ ID NO: 16 - protein, 4 aa, Homo sapiens
DIIP

SEQ ID NO: 17 - protein, 8 aa, Homo sapiens
YRELRLQR

SEQ ID NO: 18 - protein, 10 aa, Homo sapiens
YLSPYILSTL

SEQ ID NO: 19 - protein, 18 aa, Homo sapiens
RLIEDICLPRWGCLWEDD

SEQ ID NO: 20 - protein, 7 aa, Homo sapiens
HAIYPRH

SEQ ID NO: 21 - protein, 12 aa, Homo sapiens
THRPPMWSPVWP

SEQ ID NO: 22 - protein, 30 aa, Homo sapiens
GPOGPOGPOGPOGPOGPOGPOGPOGPOGPO

SEQ ID NO: 23 - protein, 14 aa, Homo sapiens
TVELQGVVPRGVNL

SEQ ID NO: 24 - protein, 8 aa, Homo sapiens
QFQSIYQY

SEQ ID NO: 25 - protein, 8 aa, Homo sapiens
VQQWRRIR

SEQ ID NO: 26 - protein, 8 aa, Homo sapiens
DLEALFYK

SEQ ID NO: 27 - protein, 6 aa, Homo sapiens
VGVAPG

(19) United States
(12) Patent Application Publication
(10) Pub. No.: US 2026/0226130 A1
(43) Pub. Date: Aug. 6, 2026

(54) ULTRA-LONG-ACTING AUTO-FUSION MAGOOLA INSULIN

(71) Applicant: DEI Biopharma, Kampala (UG)

(72) Inventor: Matthias Magoola, Kampala (UG)

(21) Appl. No.: 19/046,497

(22) Filed: Feb. 5, 2025

(51) Int. Cl. C07K 14/62

(52) U.S. Cl. CPC C07K 14/62

(57) ABSTRACT

The present disclosure provides an insulin polypeptide modification labeled as Magoola Insulin wherein insulin, proinsulin, or preproinsulin is conjugated with protein-binding peptides capable of binding to endogenous proteins including Elastin, Collagen, IgG1, IgG2, IgG4, Albumin, Transferrin, Factor XIII, IgG3, Pentameric IgM, IgA monomer, and Fibrinogen, in the body when Magoola insulin is administered to patients for long-term treatment of diabetes patients; said Magoola insulin is manufactured by recombinant technology, in vitro chemical conjugation of recombinant insulin with protein-binding peptides, or delivered using RNA technology supported by suitable pharmaceutical formulations.

### SEQUENCE LISTING

The instant application contains a Sequence Listing presented per 37 C.F.R. 1.822, which has been submitted electronically in ASCII format and is hereby incorporated by reference in its entirety. Said ASCII text file, created on Feb. 3, 2025, is named insulin.xml, and is24 kb in size.

### TECHNICAL FIELD

The present technology relates to compositions of Magoola insulin capable of binding to endogenous proteins to extend its half-life significantly, reducing dosing frequency and then delivering by RNA technology, its dose significantly.

### BACKGROUND

The following description of the background of the present technology is provided simply as an aid in understanding the present technology. It is not admitted that describes or constitutes prior art to the present technology.

During the early era of the development of biological drugs, proteins, peptides, mAbs, and vaccines, their short circulation half-lives in the human body of just a few hours were accepted as a biological fact. However, with the knowledge of IgG antibodies reaching a half-life of 1-3 weeks in humans, interest in other longer-acting biopharmaceuticals arose, appreciating that the prolonged pharmacokinetics of IgGs and their fusion proteins does not only result from their large size but also is due to the specific mechanism of FcRn-mediated endosomal recycling. It was soon discovered that an enlargement of the molecular size by way of chemically attaching polyethylene glycol (PEG) could also significantly prolong the half-life of biological drugs when conjugated to the N- or C-terminus of the peptide or protein to reduce the speed of spherical filtration and elimination of the peptide, thus increasing its plasma half-life and by slowing down renal elimination. However, using a PEG increases the risk of forming anti-PEG antibodies in vivo, which can reduce the drug's therapeutic efficacy or increase its adverse risks. The FDA has still approved several such products. Fusion proteins combining therapeutic molecules with Fc domains, albumin, or transferrin have emerged as a strategy to extend plasma half-life and improve pharmacokinetic properties. These fusion partners interact with the neonatal Fe receptor (FcRn), facilitating recycling and prolonging circulation time. Factors influencing half-life include receptor binding affinity, intracellular processing, and linker design. While Fc fusion proteins generally exhibit extended half-lives compared to their unfused counterparts, they may not achieve the longevity of full IgG molecules. Albumin fusion has shown promise in extending the half-life of various therapeutic proteins. The pharmacokinetics of peptide-Fc fusions (peptibodies) are characterized by extended half-life, renal excretion, and potential target-mediated drug disposition. Overall, fusion protein technology offers a versatile platform for improving the pharmacokinetic profiles of biotherapeutics further including Fc fusion, lipidation, albumin fusion, and many other novel approaches like biodegradable polymers and recombinant polypeptides (Table 1).

TABLE 1
Endogenous proteins and their half-lives

| | Nominal | | The ratio of | | |
|---|---|---|---|---|---|
| | Half- | | Half-Life to | | UniProt |
| | Life | Molecular | Molecular | | Accession |
| Protein | (hours) | Mass (kDa) | Mass | Half-Life Determinant | Number |
| Elastin | 682,560 | 70 | 9,750 | Extremely long-lived | P15502 |
| | | | | structural protein | |
| Collagen | 21,024 | 300 | 70 | Long-lived structure | P02452 |
| | | | | protein | |
| IgG1 | 480 | 146 | 3.3 | FcRn recycling | P01857 |
| IgG2 | 480 | 146 | 3.3 | FcRn recycling | P01859 |
| IgG4 | 480 | 146 | 3.3 | FcRn recycling | P01861 |
| Albumin | 456 | 67 | 6.8 | Size-dependent | P02768 |
| Transferrin | 288 | 80 | 3.6 | Receptor-mediated | P02787 |
| | | | | recycling | |
| Factor XIII | 168 | 320 | 0.5 | Size-dependent | P00488 |
| IgG3 | 144 | 165 | 0.87 | FcRn recycling | P01860 |
| Pentameric | 144 | 970 | 0.15 | Receptor interactions and | P01871 |
| IgM | | | | size | |
| IgA monomer | 120 | 160 | 0.75 | Receptor interactions | P01876 |
| Fibrinogen | 100 | 340 | 0.29 | Size-dependent | P02671 |
| Factor H | 87 | 155 | 0.56 | Size-dependent and | P08603 |
| | | | | complement system | |
| | | | | interactions | |
| Thyroglobulin | 65 | 660 | 0.1 | Size-dependent | P01266 |
| C-reactive | 48 | 125 | 0.38 | Size-dependent | P02741 |
| protein | | | | | |
| IgE | 48 | 188 | 0.25 | Fc receptor interactions | P01854 |
| Factor IX | 22 | 57 | 0.38 | Receptor interactions | P00740 |
| Retinol- | 12 | 21 | 0.57 | Size-dependent | P02753 |
| binding | | | | | |
| protein | | | | | |
| IFN-α | 5 | 19 | 0.26 | Receptor-mediated | P01562 |
| (Interferon- | | | | clearance | |
| alpha) | | | | | |
| Factor VIIa | 3 | 50 | 0.06 | Receptor interactions | P08709 |
| G-CSF | 2 | 20 | 0.1 | Receptor-mediated | P09919 |
| | | | | clearance | |
| IL-2 | 1.7 | 15 | 0.11 | Receptor-mediated | P60568 |
| (Interleukin- | | | | clearance | |
| 2) | | | | | |
| hGH (Human | 0.3 | 22 | 0.014 | Receptor interactions | P01241 |
| Growth | | | | | |
| Hormone) | | | | | |
| IGF-1 | 0.17 | 8 | 0.02 | Binding to IGF-binding | P05019 |
| | | | | proteins | |
| PYY3-36 | 0.13 | 4 | 0.03 | Size-dependent | P10082 |
| GLP-1 | 0.03 | 4 | 0.008 | Size-dependent | P01275 |

The endogenous proteins with long half-life can be selected to fuse with insulin; preferred examples include Elastin, Collagen, IgG1, IgG2, IgG4, Albumin, Transferrin, Factor XIII, IgG3, Pentameric IgM, IgA monomer, and Fibrinogen, each of them having their unique pharmacokinetic profile, stability and ability to interfere in the binding of insulin to receptors. These peptides can be connected with insulin as a single peptide or multiple peptides wherein each peptide is selected to connect to at least one of the selected endogenous peptide. These peptides can be connected using a flexible linker to create a single chain of peptide capable of binding to all target endogenous proteins.

### Exercise 1: Binding Interference Confirmation

To test the receptor binding possibility, insulin was designed conjugated with non-cleavable linkers, such as (G4S)n and steric hindrance examined to understand the effects of transferrin and its influence on the receptor-ligand interaction and potential therapeutic applications. The structure of the insulin-insulin receptor complex has been documented in the Protein Data Bank (PDB ID: 6PXV).

The methodology involved pre-processing and standardizing the available insulin-receptor structure using UCSF Chimera and predicting the structure of human serum transferrin attached to insulin via a (G4S)1 linker using AlphaFold3. Subsequently, insulin was docked onto the receptor's binding site using HADDOCK in its native form and after linkage with transferrin through the (G4S)1 linker. Binding affinity and interaction patterns were evaluated using the PRODIGY server, with a particular focus on the effect of the transferrin attached to insulin via a linker on interaction characteristics and steric hindrance

The results of protein-protein complex analysis revealed no significant differences in binding affinity and interaction characteristics (Table 2). For the native insulin-insulin receptor complex, the ΔG value was calculated at −16.7 kcal mol⁻¹, with a corresponding kD of 5.3E-¹³ at ° C. (Table 1). Interface analysis demonstrated a balanced contribution of polar-polar and apolar-apolar interactions within the binding pocket. Upon conjugation with transferrin via the (G4S)1 linker, we observed slightly enhanced binding affinity and similar interaction profiles. The Transferrin-(G4S)1-Insulin-Receptor complex exhibited a relatively higher ΔG value (−16.9 kcal mol⁻¹) and the lowest kD (4E⁻¹³ M), indicating stronger binding and enhanced stability. Interface analysis further revealed significant increases in polar-polar and apolar-apolar interactions for the Transferrin-(G4S)1 conjugate, while charged-charged interactions remained consistent across all models. Additionally, the conjugation of transferrin to insulin via the (G4S)1 linker would result in slower dissociation in the body, improving the pharmacokinetic profile and making it a better therapeutic product (Table 2).

TABLE 2
Binding properties of insulin with receptor when conjugated with transferrin.

| Protein- | | | ICs | ICs | ICs | ICs | ICs | ICs | | |
|---|---|---|---|---|---|---|---|---|---|---|
| protein | ΔG (kcal | | charged- | charged- | charged- | polar- | polar- | apolar- | NIS | NIS |
| complex | mol⁻¹) | K_{d} (M) | charged | polar | apolar | polar | apolar | apolar | charged | apolar |
| Insulin | −16.7 | 5.30^{E-13} | 19 | 10 | 61 | 2 | 16 | 31 | 28.54 | 34.3 |
| Receptor | | | | | | | | | | |
| Transferrin- | −16.9 | 4.00^{E-13} | 23 | 10 | 61 | 2 | 16 | 31 | 29.87 | 34.4 |
| G4S- | | | | | | | | | | |
| Insulin | | | | | | | | | | |
| receptor | | | | | | | | | | |

Transferrin naturally has a prolonged circulating half-life, ~7-10 days for the glycosylated form and 14-17 days for the non-glycosylated form. In contrast, regular insulin has a much shorter half-life, necessitating multiple daily injections for effective glucose management. By fusing insulin with transferrin, the resulting complex benefits from transferrin's prolonged half-life, allowing insulin to remain active in the body for a longer duration. A study evaluated a proinsulin-transferrin (ProINS-Tf) fusion protein for its pharmacokinetics and efficacy. The results demonstrated that ProINS-Tf exhibited a slow but sustained hypoglycemic effect in diabetic mice; specifically, after subcutaneous injection, the ProINS-Tf fusion protein-maintained blood glucose reduction for an extended period compared to regular insulin. This prolonged effect is attributed to the extended plasma half-life of the fusion protein. Furthermore, the ProINS-Tf fusion protein showed liver-specific activity, decreasing the expression of key hepatic glucose production enzymes and increasing glycogen levels. This targeted action reduces potential side effects associated with insulin therapy.

This slower dissociation is advantageous as it allows for prolonged receptor engagement, potentially reducing the frequency of administration required for effective treatment. Since our analysis indicates that the bindings between insulin and its receptor are not negatively affected by the presence of transferrin, it suggests that transferrin does not introduce any detrimental effects on receptor binding or insulin functionality. This further underscores the potential of transferrin as a carrier protein in optimizing therapeutic delivery. In previous studies, the attachment of transferrin to insulin has been shown to slow the dissociation of insulin and enhance its therapeutic effects. This is primarily due to transferrin's long circulating half-life and its ability to facilitate receptor-mediated endocytosis, which prolongs the presence of insulin in the body. This fusion approach offers a promising strategy for developing long-acting insulin therapies with improved efficacy and reduced dosing frequency, which is also supported by our results.

A critical issue for any protein therapeutic is immunogenicity. Proteins contain B and T cell epitopes, which the immune system can recognize as either self or foreign. B cell epitopes are conformational epitopes found on the surface of the proteins and are relatively difficult to assess preclinically. On the other hand, T cell epitopes are small linear peptides processed and presented by MHC to T cells. Many T-cell epitopes are known or can be predicted because of the extensive structural/functional characterization of MHC-peptide complexes. For therapeutic proteins or fusion proteins containing a native protein domain, however, the immunogenicity of the therapeutic product could potentially result in an immune response against the native protein, potentially exacerbating the disease for which the patient is being treated. Immunogenicity of fusion proteins may arise from the fusion sites (which may form neo-epitopes not recognized as self by the immune system), from allotypic responses to proteins containing allogeneic sequences, by improper formulation or packaging (which might lead to the presence of an adjuvant that helps to drive an immune response against the delivered protein, or from aggregation of the product that can help reduce the immune response.

### Fe Fusion Proteins

Fusion proteins, in general, have been known since the beginning of gene technology. The prolonged circulation of Fc fusion proteins is primarily mediated by endosomal recycling. This natural mechanism provides antibodies of the subclasses IgG1, IgG2, and IgG4 with a highly long plasma half-life of around 21 days in humans. Like antibodies, Fc fusion proteins are internalized by endothelial cells via pinocytosis, but instead of subsequent degradation in the lysosome, the Fc moiety binds to the FcRn under the slightly acidic conditions (pH≤6.5) within the endosome; subsequently, the fusion protein is transported back to the cell surface, where the complex dissociates at physiological pH≈7.4, thus releasing it into the bloodstream again. Binding target protein to immunoglobulins (IgGs) for half-life extension and sustained activity represents a promising approach, leveraging the long serum half-life of IgGs and their interaction with the neonatal Fc receptor (FcRn). Immunoglobulins are naturally long-lived in the bloodstream due to FcRn-mediated recycling, which protects them from lysosomal degradation by binding them at acidic pH in endosomes and releasing them back into circulation at neutral pH. Conjugating or genetically fusing the target protein to the Fc region of an IgG molecule allows the target protein to benefit from this recycling pathway, significantly extending its time in circulation. This strategy mimics the mechanism of Fc-fusion proteins, which are widely used in therapeutics to prolong the systemic exposure of biologics. By leveraging the Fc region's recycling properties, the half-life of the target protein can be extended from hours to several days or even weeks, which could significantly reduce the frequency of injections and improve patient compliance.

Additionally, the structural stability of the IgG backbone can protect the target protein from proteolytic degradation, enhancing its durability in the bloodstream. However, preserving the target protein's biological activity is critical, as the conjugation must avoid sterically hindering or altering the target protein's receptor-binding site. Furthermore, including an Fc region could potentially increase immunogenicity, especially if the IgG is not fully humanized or the conjugation introduces new epitopes. From a manufacturing perspective, producing the target protein-IgG conjugates pose challenges in maintaining consistency, particularly regarding the glycosylation patterns on the Fc region that are essential for interacting with FcRn. Despite these challenges, the approach is supported by the established success of Fc-fusion proteins in biopharmaceuticals, with approved drugs like etanercept and abatacept demonstrating safety and efficacy in extending half-life. Preclinical studies have also explored the target protein-Fc fusion proteins, showing prolonged glucose control in animal models and validating the potential of this strategy for human use. An alternative enhancement to using full IgGs is employing smaller engineered antibody fragments, such as single-chain variable fragments (scFvs) or nanobodies, which can retain FcRn binding while improving tissue penetration and potentially reducing immunogenicity. If these design and manufacturing considerations are carefully addressed, binding the target protein to immunoglobulins could revolutionize the target protein therapy by providing longer-lasting glucose control, reducing the burden of frequent injections, and improving patient outcomes.

A critical component of Fc fusion proteins is the linker, which can significantly affect the binding activity of the fusion partner.

Apart from prolonging the drug half-life in plasma, Fc fusion proteins can also be applied to extend ocular half-life, which was demonstrated for aflibercept (VEGF-Trap). This fusion protein comprises each extracellular domain of human VEGFR1 and VEGFR2 linked to the IgG1 Fc.

Fc fusion proteins exploit the natural recycling mechanism of the Fc receptor to prolong circulation. These proteins, which link therapeutic peptides or proteins to the Fc region of antibodies, benefit from simplified purification processes and extended half-life. Examples like etanercept and dulaglutide have demonstrated efficacy in treating chronic conditions, although issues such as reduced receptor affinity and steric hindrance can arise. Advances in Fc engineering, including the development of monovalent fusion proteins, have addressed some of these challenges, expanding their clinical utility.

FcRn is a heterodimeric receptor, closely related to major histocompatibility complex (MHC) class I receptors, which is widely expressed in vascular epithelial cells, endothelial cells, intestinal epithelial cells, mammary epithelial cells, placental membranes, monocytes, macrophages, dendritic cells, and polymorphonuclear (PMN) leukocytes. FcRn contains a 45 kDa transmembrane α-chain with a short cytoplasmic tail and a ~17 kDa β-2 microglobulin β-chain. While FcRn functions to translocate IgGs from the mother to the fetus, it also has a significant function in both IgG and HSA homeostasis. Upon pinocytosis of serum proteins by cells of the reticuloendothelial system, human IgG₁, IgG₂, and IgG₄ isotypes and HSA bind FcRn in a pH-dependent manner. As the vesicles are acidified, the IgGs and HSA bind FcRn, which allows them to be translocated back to the cell surface for recycling back into circulation. At the same time, non-FcRn-bound proteins are targeted for lysosomal degradation. Upon exposure to the neutral pH at the cell surface, the IgGs and HSA are released back into the circulation. This recycling mechanism confers a nominal 14- to 21-day half-life on human IgG₁, IgG₂, and IgG₄ and a ~19-day half-life on HSA. Human IgA, IgM, IgD, and IgE do not bind FcRn and do not possess an extended half-life. Human IgG₃ has an altered residue in the FcRn-binding domain, which decreases its ability to bind FcRn, resulting in a diminished half-life of ~5-7.5 days. The IgGs bind to FcRn at a different epitope than HSA, so the molecules do not compete. It is calculated that for every IgG molecule recycled by FcRn, ~700 molecules of HSA are recycled.

Thus, FcRn plays a significant role in the homeostasis of human IgGs and HSA, the most abundant proteins in human serum. These properties have often been used to improve the in vivo pharmacokinetics of otherwise short-lived peptides and proteins, as documented in the following sections.

Fusions of peptides and proteins can be made to human transferrin to the N- and C-termini, as well as to the centrally located hinge region that links the two major lobes of transferrin together. The N terminus of transferrin is free and can be fused directly. The C terminus is more buried and is constrained by a nearby disulfide bond, so flexible linkers are typically used when proteins are fused to the C terminus. This capability was extended by making libraries of peptides against specific targets and then fusing binders from those libraries into aglycone-transferrin (N-terminal, C-terminal, loops, or linker region) to be developed into therapeutic fusion proteins with extended half-lives.

### Albumin

Human serum albumin (HSA), a 66.5 kDa protein produced in huge quantities by hepatocytes (~14 g per day), is the most abundant protein in the blood, with a concentration between 35 and 50 mg/mL. In the blood plasma, HSA not only acts as ampholyte and osmolyte but also as a carrier for many physiologically relevant compounds such as hormones (progesterone, testosterone, thyroid hormones, etc.), bilirubin and, notably, various fatty acids. Its exceptionally long plasma half-life of 19 days in humans is in part related to a reduced renal clearance due to the large molecular size as well as the net negative charge at physiological pH (pI 5.9), even though around 3.3 g of albumin is still lost by kidney filtration every day. However, the main reason for its long circulation is endosomal recycling since HSA binds to FcRn similarly to Igs but at an interface that differs from one of the Fc portions. In contrast, albumin does not interact with Fcγ receptors and, consequently, does not activate immune cells. One strategy to further improve the half-life of albumin fusion proteins is the engineering of HSA variants for increased FcRn affinity.

Albumin fusion and albumin-binding technologies continue to evolve. These strategies provide significant half-life extension by leveraging albumin's inherent recycling properties through Fc receptor binding. Albiglutide and albutrepenonacog alfa are examples of successful applications, though competition with endogenous albumin can limit drug efficacy. Innovations in albumin engineering, such as modifying albumin for increased receptor affinity, show potential for further improvements in circulation time and therapeutic performance.

Albumin is a widely used carrier protein for enhancing the half-life of therapeutic proteins due to its natural abundance, long half-life (~19-21 days), and ability to bind various ligands. Its interaction with the neonatal Fc receptor (FcRn) facilitates this property, which protects albumin from lysosomal degradation, thus extending its circulation time. Albumin binding is typically achieved through direct conjugation or fusion technologies, and several therapeutic proteins have leveraged this strategy. For example, albiglutide, an albumin-fused GLP-1 receptor agonist used for treating type 2 diabetes, demonstrates a significantly prolonged half-life compared to unmodified GLP-1. Similarly, certolizumab pegol, a PEGylated Fab fragment that indirectly benefits from albumin binding, is designed to enhance half-life and efficacy in autoimmune diseases. Albumin-binding peptides have also been incorporated into drug designs to exploit this natural recycling mechanism. In mRNA therapeutics, albumin fusion proteins expressed from mRNA have been explored preclinically, aiming to achieve sustained expression of therapeutic proteins while minimizing frequent dosing requirements. Still, no product has yet reached clinical approval. These applications highlight the versatility of albumin in improving pharmacokinetics and reducing administration frequency for various therapies. An example is albiglutide, an albumin-fused GLP-1 receptor agonist, which has demonstrated prolonged activity in type 2 diabetes by reducing the need for frequent administration.

### ELPs

Other innovative strategies include elastin-like polypeptides (ELPs), which undergo reversible phase transitions to form drug depots, allowing sustained release. In a different approach, artificial elastin-like polypeptides (ELPs) are constructed based on Val-Pro-Gly-Xaa-Gly pentapeptide repeats, where Xaa can be any amino acid except for Pro. The unique feature of ELPs is that they can undergo a reversible temperature-dependent phase transition that is governed by the residue at the Xaa position. Thus, ELPs have been engineered to be soluble at room temperature but to aggregate at body temperature, forming a gel-like state. Similarly, elastin-like polypeptides (ELPs) provide a unique mechanism of sustained release through temperature-dependent depot formation, offering a promising option for extended dosing intervals.

As of now, there are only drug FDA-approved therapeutics Glymera™ (PB1023), a recombinant GLP-1 analog intended for the treatment of conditions such as sarcopenia, fused to elastin-like peptide (ELP) giving a half-life of ~36 hours extended from 1-2 minutes for the active GLP-1. In May 2023, Duke University licensed this drug development related to ELP platform technology. Another drug, PB1046, is a once-weekly fusion protein designed to treat pulmonary arterial hypertension (PAH). PB1023 is a long-acting drug.

Binding the target protein with elastin-like polypeptides (ELPs) or elastin-like sequences can significantly enhance the half-life of the target protein and potentially sustain its activity for weeks or even months. This approach leverages the unique properties of elastin-like sequences, such as their thermoresponsive behavior, prolonged systemic circulation, and ability to form depots in physiological conditions. When conjugated to the target protein, these sequences exhibit thermosensitivity, allowing them to create a gel or depot at body temperature, facilitating the target protein's slow and sustained release over an extended period. By increasing the molecular weight of the target protein, elastin-like polypeptides also reduce renal clearance, prolonging its presence in the bloodstream. Furthermore, these sequences provide a protective shield against proteolytic enzymes, enhancing the target protein's stability and bioavailability. Upon injection, the elastin target protein conjugate forms a localized depot at the injection site, enabling gradual release that could match the body's physiological demand for the target protein.

The feasibility of this method depends on ensuring that the target protein remains biologically active after release and that the conjugation does not interfere with its receptor-binding domain. Studies, such as those exploring the depot-forming capabilities of elastin-like polypeptides, have shown promising results, with sustained release profiles and extended glucose control in preclinical models. However, challenges remain, including rigorous testing to confirm that the conjugate remains intact and active without significant degradation over long periods, the risk of immune reactions or local inflammation, and the complexity of large-scale manufacturing to ensure consistent quality. While regulatory hurdles must be addressed, this approach can potentially revolutionize target protein therapy by reducing the need for frequent injections and improving patient compliance, representing a significant advancement in diabetes management.

### Collagen

For collagen, the potential benefits are tied to its structural and depot-forming properties. Collagen is a major extracellular matrix protein that is stable and slowly degraded in the body. Binding the target protein to collagen could allow the formation of a depot-like structure at the injection site, from which the target protein could be gradually released. This approach suits subcutaneous or localized delivery systems where collagen's natural resilience and biocompatibility could sustain the target protein release. However, challenges include ensuring that the collagen-target protein conjugate does not interfere with collagen's natural fibril formation or lead to fibrosis or inflammation at the administration site. Additionally, the molecular interaction between the target protein and collagen must preserve the target protein's receptor-binding capability to ensure its bioactivity upon release.

While both elastin and collagen can form depots to prolong the target protein's activity, the mechanisms differ. Elastin relies on thermoresponsive behavior and depot dissolution, while collagen depends on matrix stability and enzymatic degradation to control the target protein release. These differences would influence each system's release profile, biocompatibility, and potential applications in therapeutic target protein delivery.

### Transferrin

Transferrin is a highly abundant serum glycoprotein, found in serum at 3-4 mg/mL, which binds iron tightly but reversibly and functions to carry iron to tissues. Transferrin has 679 amino acid residues, is about 80 kDa in size, and possesses two high-affinity Fe³⁺-binding sites, one in the N-terminal domain and the other in the C-terminal domain. Human transferrin has a half-life of 7-12 days. The aglycosylated form of human transferrin, which makes up about 2-8% of the total transferrin pool, has a slightly longer half-life of 14-17 days. The extended persistence of transferrin in human serum is due to a clathrin-dependent transferrin receptor-mediated mechanism, which recycles transferrin receptor-bound transferrin back into circulation.

Transferrin binds and transports iron and has a half-life of ~8 days. This longevity is crucial for maintaining iron homeostasis and ensuring that iron is delivered efficiently to tissues without being lost through renal clearance. The tight binding of transferrin to iron also protects it from proteolytic degradation, enhancing its stability in circulation.

Similarly, transferrin and the Fc portion of immunoglobulins offer unique advantages in half-life extension and targeted delivery. Transferrin binds to cell surface receptors and undergoes recycling via endosomes, providing prolonged circulation and targeted delivery to tissues expressing transferrin receptors. A flexible linker ensures the therapeutic protein remains tethered to transferrin without steric interference, maintaining receptor interactions. In contrast, the Fc region of immunoglobulins offers exceptional systemic stability, with a half-life exceeding 20 days due to FcRn-mediated recycling. The Fc carrier prolongs systemic circulation while preserving the accessibility of the therapeutic protein for receptor interactions. While transferrin excels in tissue-specific delivery through receptor-mediated pathways, the Fc region focuses on systemic stability, making each carrier suitable for therapeutic applications. The careful design of the linker in both cases ensure minimal disruption to the carrier protein's natural recycling or transcytosis mechanisms.

In addition to half-life extension, conjugating insulin or other therapeutic proteins to transcytosis-enabled carriers, such as albumin or transferrin, can expand distribution volume by facilitating transport across cellular barriers like the blood-brain barrier or endothelial linings. This mechanism enhances tissue penetration and delivery to otherwise restricted compartments, leveraging the carrier's receptor-mediated endocytosis and transcytosis. However, the half-life extension relies more on the carrier's recycling properties, such as FcRn-mediated protection from degradation, rather than on transcytosis itself. Transferrin primarily enhances tissue-specific delivery, while albumin and Fc carriers combine extended systemic presence with broader tissue access. Designing conjugates that integrate transcytosis and recycling mechanisms allows for optimized pharmacokinetics and pharmacodynamics, maximizing therapeutic efficacy through expanded tissue penetration and prolonged systemic durability. These approaches require rigorous evaluation to balance the complementary roles of half-life extension and distribution volume in achieving desired therapeutic outcomes.

### Thyroxine Binding Globulin

Proteins such as TBG (thyroxine-binding globulin) exhibit extended half-lives due to their specific and high-affinity binding to ligands. TBG binds thyroid hormones (e.g., thyroxine and triiodothyronine) with high affinity, extending their half-life and regulating their availability to tissues. This tight binding ensures a steady supply of thyroid hormones to target organs, allowing precise control of metabolic processes. TBG's extended half-life, combined with its role as a reservoir for thyroid hormones, exemplifies how tight ligand binding can protect proteins from rapid turnover.

TBG is a carrier protein produced by the liver that binds and transports thyroid hormones, triiodothyronine (T3) and thyroxine (T4), in the bloodstream, regulating their availability and preventing rapid clearance. In contrast, TSH is a hormone secreted by the pituitary gland that stimulates the thyroid gland to produce and release T4 and T3, playing a central role in the feedback loop that regulates thyroid hormone levels. High TSH levels indicate hypothyroidism, while low levels suggest hyperthyroidism. When considering strategies to extend the half-life of a therapeutic protein, binding to a carrier protein like TBG can be advantageous because it creates a reservoir of the hormone that is gradually released into circulation. This mechanism mimics natural thyroid hormone transport and reduces the frequency of administration. However, for therapeutic proteins in general, the choice of an optimal carrier depends on factors such as receptor affinity, clearance mechanisms, and potential immunogenicity. Other strategies, such as fusion to albumin or Fc fragments, may offer greater stability and lower immunogenic risk, depending on the specific therapeutic application.

### Thyroid Stimulating Hormone (TSH)

Another interesting peptide fusion approach has a very different mechanism of action. Thyroid-stimulating hormone (TSH; also known as thyrotropin) and the three gonadotropins, follicle-stimulating hormone (FSH), luteinizing hormone (LH), and CG, are heterodimeric glycohormones consisting of a common α-subunit and unique β-subunits, which confer on them their different activities. The half-life of human CG (HCG) is significantly longer than that of its counterparts, FSH, LH, and TSH. The difference between HCG and its counterparts is that the HCG β-subunit (HCG-0) possesses a ~31-amino-acid-residue CTP consisting of the sequence FQSSSS*KAPPPS*LPSPS*RLPGPS*DTPILPQ, which possesses four O-glycosylation sites (denoted by S*) terminating with a sialic acid residue. CTP has been demonstrated to naturally extend that protein's half-life in human serum, likely because the negatively charged, heavily sialylated CTP impairs renal clearance.

### Protein-Binding Peptides

Synthetic peptides that bind to albumin, Fc regions, or transferrin receptors can be strategically designed to improve the pharmacokinetics, stability, and targeting of therapeutic proteins like insulin. Albumin-binding peptides leverage albumin's natural abundance and long half-life (~19 days) to extend the circulation time of therapeutics. For example, peptides like SAFF-1 (LRHYY), derived from bacterial albumin-binding proteins, and DKFYW, designed for strong albumin interaction, can be short, specific, and minimally immunogenic. Fatty acid-modified peptides, such as those conjugated with palmitic acid, mimic natural fatty acid binding to albumin's hydrophobic pockets. Other peptides like DIIE and DIIP provide similar binding capabilities and have been used for pharmacokinetic enhancements.

Fc-binding peptides can be engineered to bind to the Fc regions of IgG antibodies, leveraging neonatal Fc receptor (FcRn)-mediated recycling to extend the half-life of therapeutic proteins. Examples include FcBP1 (QFQSIYQY), which is small, stable, and highly specific to the Fc region, and AbFc (VQQWRRIR), another Fc-binding peptide with high specificity. These peptides can be integrated into fusion constructs with therapeutic proteins to enhance stability and circulation time.

Transferrin-binding peptides target the transferrin receptor (TfR), facilitating receptor-mediated endocytosis and tissue-specific delivery. The T7 peptide (HAIYPRH) is a widely used short sequence that binds TfR1 with high specificity and is particularly effective in enhancing uptake into tissues like the liver and muscle. Other examples include TfR-BP9 (THRPPMWSPVWP), a longer peptide with a high affinity for TfR, and IRL-1803 (TRP), a minimal motif for transferrin receptor interaction. These peptides can be employed in drug delivery systems for cancer therapy and targeted therapeutics.

### Collagen Hybridizing Peptide (CHP)

The Collagen Hybridizing Peptide (CHP) is most effective when designed to mimic the triple-helix structure of native collagen, enabling it to hybridize with denatured collagen strands. Studies indicate that at least 6-9 Gly-Pro-Hyp (GPO) repeats, corresponding to 18-27 amino acids, are necessary for stable binding, as shorter peptides may lack the structural integrity required for effective hybridization. For enhanced stability and affinity, 9-12 GPO repeats (27-36 amino acids) are typically used, providing stronger and more durable interactions with collagen. Increasing the length beyond 12 repeats may offer marginal improvements in thermostability but does not significantly enhance binding affinity. Additionally, cyclic CHP structures can further improve binding by pre-organizing the peptide into a helical conformation. Overall, a CHP of at least 6-9 GPO repeats is required for effective binding, with 9-12 repeats being optimal for robust and stable collagen interactions.

To implement such peptides in a therapeutic fusion construct, linkers like (GGGGS)n can be used to ensure flexibility and functional independence, minimizing steric hindrance. For example, insulin fused with SAFF-1 for albumin binding, FcBP1 for Fc region recycling, and T7 for TfR targeting, connected via flexible linkers, could significantly enhance stability and tissue-specific delivery while preserving insulin's receptor-binding function. Computational tools like AlphaFold and molecular docking software optimize the construct's design, ensuring spatial separation of functional domains and high binding affinity to target proteins. This modular approach is validated through in vitro assays such as SPR and ITC for binding evaluation and in vivo studies for pharmacokinetics and biodistribution (Table 3)

TABLE 3
Short peptides to connect serum albumins

| Target | Peptide | | | |
|---|---|---|---|---|
| Protein | Name | Sequence | Length | Notes |
| Albumin | SAFF-1 | LRHYY | 5 | Minimal immunogenicity; highly |
| | | (SEQUENCE | | specific. |
| | | NO 14) | | |
| Albumin | DIIE | DIIE | 4 | Derived from bacterial albumin- |
| | | (SEQUENCE | | binding domains. |
| | | NO 15) | | |
| Albumin | DIIP | DIIP | 4 | Alternative to DIIE with similar |
| | | (SEQUENCE | | properties. |
| | | NO 16) | | |
| Albumin | YRELRLQR | YRELRLQR | 8 | High-affinity albumin binder. |
| | | (SEQUENCE | | |
| | | NO 17) | | |
| Albumin | YLSPYILSTL | YLSPYILST | 10 | Engineered for strong binding. |
| | | L | | |
| | | (SEQUENCE | | |
| | | NO 18) | | |
| Albumin | SA21 | Ac- | 19 | Identified via phage display; binds |
| | | RLIEDICLP | | albumin with high affinity. |
| | | RWGCLWE | | |
| | | DD-NH₂ | | |
| | | (SEQUENCE | | |
| | | NO 19) | | |
| Transferrin | T7 | HAIYPRH | 7 | Widely used in targeting TfR- |
| Receptor | | (SEQUENCE | | expressing cells. |
| | | NO 20) | | |
| Transferrin | TfR-BP9 | THRPPMW | 12 | High affinity for TfR; longer |
| Receptor | | SPVWP | | peptide. |
| | | (SEQUENCE | | |
| | | NO 21) | | |
| Transferrin | IRL-1803 | TRP | 3 | Minimal binding motif for transferrin |
| Receptor | | (SEQUENCE | | receptor. |
| | | NO 22) | | |
| Collagen | Collagen | GPOGPOGP | 30 | Mimics collagen triple helix; binds |
| | Hybridizing | OGPOGPOG | (Variable) | denatured collagen strands. |
| | Peptide | POGPOGPO | | |
| | (CHP) | GPOGPO | | |
| | | (SEQUENCE | | |
| | | NO 23) | | |
| Factor XIII | Factor XIII | TVELQGVV | 14 | The activation peptide is cleaved by |
| | Activation | PRGVNL | | thrombin to activate Factor XIII. |
| | Peptide | (SEQUENCE | | |
| | | NO 24) | | |
| Fc Region | FcBP1 | QFQSIYQY | 8 | Enhances half-life via FcRn |
| (IgG) | | (SEQUENCE | | recycling. |
| | | NO 25) | | |
| Fc Region | AbFc | VQQWRRIR | 8 | Strong Fc-binding activity. |
| (IgG) | | (SEQUENCE | | |
| | | NO 26) | | |
| FcRn | FcRn-BP | DLEALFYK | 8 | Used for Fc recycling applications. |
| Receptor | | (SEQUENCE | | |
| | | NO 27) | | |
| Elastin | VGVAPG | VGVAPG | 6 | Repeats in tropoelastin; involved in |
| | | (SEQUENCE | | elastic fiber formation. |
| | | NO 28) | | |
| Fibrinogen | RGD Peptide | RGD | 3 | Binds to integrin receptors; involved |
| | | (SEQUENCE | | in cell adhesion. Binding with |
| | | NO 29) | | Fibrinogen |

TABLE 4
Fusion proteins approved by the FDA with half-life extension

| | | Half-Life of | | Half-Life of |
|---|---|---|---|---|
| | | Fusion | | Unconjugated |
| Fusion Protein | Components | Protein | | Protein |
| Alprolix ® | Factor IX fused to the Fc | 57-83 | hours | Factor IX: 18-24 |
| (eftrenonacog-α) | region of human IgG₁ | | | hours |
| Arcalyst ® | IL-1 receptor components | 7 | days | IL-1 receptor |
| (rilonacept) | fused to the Fc region of | | | antagonist: 4-6 hours |
| | human IgG₁ | | | |
| Eloctate ® | B-domain-deleted Factor | ~19 | hours | Factor VIII: 8-12 |
| (efraloctocog-α) | VIII fused to the Fc region | | | hours |
| | of human IgG₁ | | | |
| Elonva ® | Follicle-stimulating hormone | ~65 | hours | FSH: 3-4 hours |
| (corifollitropin-α) | (FSH) fused to carboxy- | | | |
| | terminal peptide (CTP) of | | | |
| | human chorionic | | | |
| | gonadotropin | | | |
| Enbrel ® (etanercept) | Tumor necrosis factor | ~4 | days | Soluble TNFR: |
| | receptor (TNFR) fused to the | | | Minutes to hours |
| | Fc region of human IgG₁ | | | |
| Eylea ® (aflibercept) | Vascular endothelial growth | ~5-6 | days | Soluble VEGF |
| | factor (VEGF) receptor | | | receptor: Hours |
| | fused to Fc region of human | | | |
| | IgG₁ | | | |
| Factor IX-HSA | Factor IX Padua variant | 7.8-11.1 | | Factor IX: 18-24 |
| Fusion Protein | fused to human serum | days (in | | hours |
| | albumin | animal | | |
| | | studies) | | |
| G-CSF-HSA Fusion | Granulocyte colony- | 9.3 hours (in | | G-CSF: 3.5-3.8 hours |
| Protein | stimulating factor (G-CSF) | animal | | |
| | fused to human serum | studies) | | |
| | albumin | | | |
| Glymera ™ (PB1023) | GLP-1 fused to elastin-like | ~36 | hours | Native GLP-1: 1-2 |
| | peptide (ELP) | | | minutes |
| Nplate ® | Thrombopoietin mimetic | 3.5 (1-34) | | Native |
| (romiplostim) | peptide fused to Fc region of | days | | thrombopoietin: 30 |
| | human IgG₁ | | | hours |
| Orencia ® (abatacept) | CTLA-4 fused to Fc region | 13-16 | days | CTLA-4: Not |
| | of human IgG₁ | | | specified |
| Tanzeum ®/Eperzan ® | GLP-1 fused to human | 4-7 | days | Native GLP-1: 1-2 |
| (albiglutide) | serum albumin (HSA) | | | minutes |
| Trulicity ® | GLP-1 fused to Fc region of | 4-5 | days | Native GLP-1: 1-2 |
| (dulaglutide) | human IgG₄ | | | minutes |
| VRS-859 (exendin 4- | Exendin-4 fused to XTEN | 1 | month | Exendin-4: 2.4 hours |
| XTEN) | polypeptide | | | |
| Zaltrap ® (ziv- | VEGF receptor fused to Fc | ~6 | days | Soluble VEGF |
| aflibercept) | region of human IgG₁ | | | receptor: Hours |

Binding non-covalently, via a peptide or protein-binding domain attachment to the bioactive protein, to normally long-half-life proteins such as HSA, human IgG, or possibly transferrin. However, it is reported that the in vitro biological activity of fusion proteins, as measured by the half-maximal inhibitory concentration (IC₅₀) or the half-maximal effective concentration (EC₅₀), is often lower than that of the original biologic that has been fused to a protein to elongate its pharmacokinetic profile. These losses in activity may be due to the decrease in the on-rate due to the bulkiness of the fusion protein as compared with the original bioactive protein or steric hindrance. It has been demonstrated that the linker type, length, and flexibility, as well as the fusion of the bioactive peptide or protein to the C or N terminus of the half-life-extension module, can profoundly affect the activity of the fusion proteins. Examples follow for this phenomenon and how protein engineering can sometimes overcome this loss of activity. It is imperative, therefore, to test the impact of fusion before deciding the proposed structure of fusion proteins.

### Linkers

In some examples, the C-terminus of the insulin polypeptide is connected directly to the N-terminus of the long half-life endogenous proteins (e.g., no linker or linker absent). In other examples, successfully constructing a recombinantly made Magoola insulin requires a linker connecting the insulin polypeptide to the long half-life endogenous proteins. In embodiments, Magoola insulin configurations described herein comprise a peptide linker between the insulin polypeptide and the long half-life endogenous proteins composed of amino acids (e.g., natural or unnatural amino acids). In embodiments, the peptide linker can be encoded by a nucleic acid molecule, for example, such that a single nucleic acid molecule can encode the various peptides within an insulin polypeptide as well as the peptide linker and the long half-life endogenous proteins. The choice of peptide linker (for example, the length, composition, hydrophobicity, and secondary structure) could impact the manufacturability of the Magoola insulin configuration (i.e., the homodimer titer), the chemical and enzymatic stability, the bioactivity (i.e., the NAOC value), parameters that correlate with bioactivity, and the immunogenicity of the insulin-Fc fusion protein. Several linkers can be used in designing Magoola insulin configurations to improve the homodimer titer and the bioactivity.

### DETAILED DESCRIPTION

Diabetes is a chronic condition characterized by an insulin deficiency and/or ineffective use of insulin. People with diabetes who have an absolute deficiency of insulin are categorized as having type 1 or insulin-dependent diabetes mellitus (IDDM). Type 1 diabetics are thought to have a genetic predisposition combined with immunologic destruction of the insulin-producing 3-cells of the pancreas. In comparison, people with diabetes who can still produce some insulin but have a relative deficiency due to insulin resistance or other dysfunction are classified as having type 2 or non-insulin-dependent diabetes mellitus (NIDDM). Type 2 diabetes is linked to genetic predisposition, obesity, and certain medications. Women can also develop temporary insulin resistance during pregnancy in what is called gestational diabetes. Some adults are diagnosed with latent autoimmune diabetes in adults (LADA), a slow-progressing form of autoimmune diabetes. Similar to type 1 diabetes, in LADA, the insulin-producing (3-cells of the pancreas are destroyed but at a slower rate. A small percentage of people are diagnosed with maturity-onset diabetes of the young (MODY), which refers to any of several hereditary forms of diabetes mellitus caused by mutations in an autosomal dominant gene disrupting insulin production.

When a type 1 diabetes, LADA, or MODY patient's pancreas does not produce enough insulin, the patient generally exhibits an atypical glycemia phenotype marked by hyperglycemia. In these cases, the patients are treated with chronic insulin injection therapy. In type 2 and gestational diabetes, patients also often exhibit hyperglycemia as they are unable to properly utilize the insulin that is being produced by the pancreas. In these cases, the patients can be treated with oral medication with or without changes in diet and exercise; however, many subjects eventually progress to resemble a type 1 diabetes condition (an inflammatory disease in the pancreas with significant loss of beta cell mass) and become dependent on exogenous insulin. Left untreated, diabetes can lead to weight loss, loss of appetite, vomiting, dehydration, problems with motor function, coma, and even death.

Approximately 30 million people, or 9.4% of the United States population, have diabetes. Type 1 diabetes accounts for about 5% of all diagnosed cases of diabetes, affecting approximately 1.5 million people. Current diabetes therapies include various short-acting and long-acting insulin products administered via subcutaneous injection multiple times daily or through a wearable subcutaneous infusion pump. The burden of frequent injections results in a lack of treatment regimen compliance and under-dosing, leading to poor long-term health outcomes. Each year, over 7 million hospital discharges related to diabetes are reported among United States adults due to cardiovascular events, amputations, and ketoacidosis.

Furthermore, each year, over 14 million emergency department visits related to diabetes are reported among United States adults due to hypoglycemia and hyperglycemia crises, among other conditions. Among U.S. adults aged 20 years or older with diagnosed diabetes, the estimated prevalence of kidney disease is over 36%. Diabetes is the seventh leading cause of death in the United States, with a total estimated annual cost of over $245 billion. Therefore, there is a need for cost-effective and less burdensome treatment options for this disease.

Fusion technology has emerged as a promising approach to extend the half-life of insulin and other antidiabetic peptides. Various strategies have been explored, including fusion with transferrin, albumin, and immunoglobulin Fc regions. These fusion proteins demonstrate prolonged plasma half-lives, ranging from hours to days, compared to minutes for native peptides. Conjugation with antithrombin III-binding pentasaccharides has also shown potential in extending half-life. Additionally, fusion with albumin-binding aptides has been investigated. These approaches increase the duration of action and maintain or enhance the pharmacological effects of the original peptides. The extended half-life allows for less frequent dosing, potentially improving patient compliance and treatment outcomes in diabetes management (Table 6)

TABLE 6
Insulin analogs

| Insulin Analog/Fusion | | |
|---|---|---|
| Protein | Modification/Conjugation | Pharmacokinetic Impact |
| Human Insulin | None. Half-life 5-7 minutes | Rapidly cleared from |
| | | circulation; necessitates |
| | | multiple daily injections. |
| Insulin Aspart | Substitution of proline (B28) | Rapid-acting insulin, faster |
| | with aspartic acid for faster | onset, and shorter duration |
| | absorption | |
| Insulin Glargine U100 | Addition of two arginine | Long-acting insulin, forms |
| | residues (B31, B32) and | micro precipitates for slow |
| | replacement of asparagine | release (~24 hours) |
| | (A21) with glycine for pH- | |
| | dependent precipitation | |
| Insulin Detemir | Conjugation with myristic | Long-acting insulin binds |
| | acid (C14) at lysine B29 for | albumin for prolonged |
| | albumin binding | absorption (~20 hours) |
| Insulin Icodec | Conjugation with C20 fatty | Ultra-long-acting insulin, |
| | diacid via a linker at lysine | reversible albumin |
| | B29 for ultra-long duration | binding, ~196-hour half-life |
| BIF (Basal Insulin Fc) | Fusion with an Fc fragment | Ultra-long-acting insulin with |
| | of IgG to reduce insulin | reduced IR binding, designed |
| | receptor (IR) binding while | for once-weekly dosing (~1- |
| | significantly prolonging half- | week half-life) |
| | life via FcRn recycling | |
| Insulin-Fc Fusion Protein | Fusion with an Fc fragment | Prolonged insulin action via |
| | of IgG, maintaining normal or | Fc fusion increased molecular |
| | near-normal insulin receptor | weight and reduces renal |
| | (IR) binding, leading to | clearance, but IR binding |
| | prolonged action and reduced | varies depending on fusion |
| | renal clearance | design. Estimated half- |
| | | life: ~3-7 days. U.S. Pat. |
| | | No. 11,352,407 |
| Insulin Degludec | Attachment of | Ultra-long-acting insulin |
| | hexadecanedioic acid to | forms a subcutaneous depot |
| | lysine at position B29, | duration of action of up to 42 |
| | allowing multi-hexamer | hours |
| | formation | |
| Proinsulin-Transferrin | Fusion of proinsulin with | Long-acting insulin with |
| Fusion Protein | transferrin to increase | sustained in vivo |
| | molecular size and utilize | hypoglycemic efficacy and |
| | transferrin receptor pathways | extended plasma half-life; |
| | | 7.29 hours following |
| | | subcutaneous administration. |
| | | This extended half-life |
| | | contributed to a sustained |
| | | hypoglycemic effect in the |
| | | test subjects. |

For a selected dosing interval, such as once a week, the primary consideration is that these new formulations maintain a consistent pharmacokinetic and pharmacodynamic profile over a more extended period. Any fluctuation in insulin levels could lead to hyperglycemia or hypoglycemia, both of which are dangerous for patients with diabetes. While glycemic control is the primary endpoint in most current studies, diabetes is a systemic disease that involves multiple organ systems. Hence, it is important to understand how weekly insulin treatment might moderate these broader health outcomes.

An insulin treatment requiring less frequent dosing (e.g., once-weekly or once-monthly injections) would be less burdensome on patients, leading to better compliance, glucose control, and ultimately better long-term health outcomes. As disclosed herein, proposed ultra-long-acting insulin treatments for human clinical use comprise a Magoola insulin using a human long half-life endogenous protein to prolong their action in vivo. A Magoola insulin suitable for an ultra-long-acting treatment for diabetes should meet various design goals. A Magoola insulin suitable for an ultra-long-acting therapy for diabetes can be manufactured by any recombinant technology, chemically conjugating insulin with protein-binding peptides or RNA technology.

The Magoola insulin configuration also exhibits sustained bioactivity in vivo (e.g., demonstrate glucose lowering activity greater than about a week, month of even a year to justify less frequent dosing. The sustained bioactivity and prolonged residence time of a given Magoola insulin configuration may be predicted by its ability to bind the receptors responsible for the prolonged in vivo elimination half-lives.

Proposed ultra-long-acting insulin treatments for human clinical use comprise a Magoola insulin making use of human long-life endogenous proteins to prolong their action in vivo. Magoola insulin configurations that are useful as ultra-long-acting insulins, and they are not expected to be more immunogenic than any other current means of administering insulin.

Approved long-acting insulin analogs for basal insulin therapy form a subcutaneous depot at the injection site (insulin glargine, detemir) and/or exhibit an extended half-life through hydrophobic interaction of fatty acid groups with serum albumin (insulin detemir). Both detemir and glargine require twice-daily administration. The binding of a polypeptide drug (either covalently or through a tightly binding carrier) to long-lived plasma proteins in the bloodstream enables tight control over targeted drug levels with improved exposure and distribution in the circulation that may potentially lead to lower doses, decreased side effects, and enhanced predictability.

### SUMMARY OF THE PRESENT TECHNOLOGY

While several technologies have been introduced to extend the half-life of insulin, in one aspect, the present disclosure provides a fusion protein forming upon entry of Magoola insulin polypeptide that is conjugated with a protein-binding peptide or multiple protein-binding peptides capable of binding with long half-life endogenous proteins, albumin, transferrin, elastin, collagen, Factor XIII or thrombin, or a combination thereof. These protein-binding peptides are connected by a flexible linker, providing a sequence structure of Magoola insulin, as shown in Table 7.

TABLE 7
Sequence design of Magoola insulin

| Description | Segment |
|---|---|
| Proinsulin with | MALWMRLLPLLALLALWGPDPAAAFVNQHLCGSHLVEALYLVCGE |
| signal peptide | RGFFYTPKTRREAEDLQVGQVELGGGPGAGSLQPLALEGSLQKRGIV |
| | EQCCTSICSLYQLENYCN (SEQ NO 1) |
| Flexible Linker | GGGGS (SEQUENCE NO 2), GGGGAGGGG (SEQUENCE NO 3), |
| | GGGGSGGGG (SEQUENCE NO 4), GGGGGAGGGG (SEQUENCE NO 5), |
| | GGGGSGGGGSGGGGSGGGG (SEQUENCE NO 6), |
| | GGGGKGGGGKGGGGKGGGG (SEQUENCE NO 7), |
| | GGGGGAGGGGAGGGGAGGGGG (SEQUENCE NO 8), |
| | GGGGGQGGGGQGGGGQGGGGG (SEQUENCE NO 9), |
| | SGGGGQGGGGQGGGGQGGGGG (SEQUENCE NO 10), |
| | HGGGGQGGGGQGGGGQGGGGG (SEQUENCE NO 11), |
| | PGGGGGQGGGGQGGGGQGGGGG (SEQUENCE NO 12), |
| | GGGGGQGGGGQGGGGQGGGGGQGGGG (SEQUENCE NO 13) |
| Protein-binding- | LRHYY (SEQUENCE NO 14), DIIE (SEQUENCE NO 15), DIIP |
| peptide | (SEQUENCE NO 16), YRELRLQR (SEQUENCE NO 17), YLSPYILSTL |
| | (SEQUENCE NO 18), RLIEDICLPRWGCLWEDD (SEQUENCE NO 19; |
| | Ac-RLIEDICLPRWGCLWEDD-NH₂), HAIYPRH (SEQUENCE NO 20), |
| | THRPPMWSPVWP (SEQUENCE NO 21), |
| | GPOGPOGPOGPOGPOGPOGPOGPOGPOGPO (SEQUENCE NO 22), |
| | TVELQGVVPRGVNL (SEQUENCE NO 23), QFQSIYQY (SEQUENCE |
| | NO 24), VQQWRRIR (SEQUENCE NO 25), DLEALFYK (SEQUENCE |
| | NO 26), VGVAPG (SEQUENCE NO 27), TRP (SEQUENCE NO 28), RGD |
| | (SEQUENCE NO 29) |

To ensure the protein-binding peptide remains attached after proinsulin processing, it must be placed outside the C-peptide region, which is enzymatically removed during insulin maturation. The two main locations for conjugation are at the N-terminal of the B-chain or at the C-terminal of the A-chain.

By adding the protein-binding peptide at the start of the B-chain, insulin's overall function is minimally affected, and it remains albumin-bound post-cleavage. This placement allows for extended circulation of insulin without interfering with insulin receptor binding. The sequence for this modification is: Protein-Binding-Peptide-Proinsulin

Alternatively, the protein-binding peptide can be placed at the end of the A-chain, ensuring it remains part of the mature insulin molecule. This placement preserves normal insulin folding and maintains full biological activity, while still allowing albumin binding. The sequence is: Proinsulin-linker-Protein-Binding-Peptide

In some examples, the C-terminus of the insulin polypeptide is connected directly to the N-terminus of the long half-life endogenous proteins (e.g., no linker or linker absent). In other examples, successfully constructing a recombinantly made Magoola insulin requires a linker connecting the insulin polypeptide to the long half-life endogenous proteins. In embodiments, Magoola insulin configurations described herein comprise a peptide linker between the insulin polypeptide and the long half-life endogenous proteins composed of amino acids (e.g., natural or unnatural amino acids). In embodiments, the peptide linker can be encoded by a nucleic acid molecule, for example, such that a single nucleic acid molecule can encode the various peptides within an insulin polypeptide as well as the peptide linker and the long half-life endogenous proteins. The choice of peptide linker (for example, the length, composition, hydrophobicity, and secondary structure) could impact the manufacturability of the Magoola insulin configuration (i.e., the homodimer titer), the chemical and enzymatic stability, the bioactivity (i.e., the NAOC value), parameters that correlate with bioactivity, and the immunogenicity of the insulin-Fc fusion protein. Table 4 lists several linkers used in designing Magoola insulin configurations to improve the homodimer titer and the bioactivity.

### DETAILED DESCRIPTION

An insulin treatment requiring less frequent dosing (e.g., once-weekly or once-monthly injections) would be less burdensome on patients, leading to better compliance, glucose control, and ultimately better long-term health outcomes. As disclosed herein, proposed ultra-long-acting insulin treatments for human clinical use comprise a Magoola insulin using a human long half-life endogenous protein to prolong their action in vivo. A Magoola insulin suitable for an ultra-long-acting treatment for diabetes should meet various design goals. A Magoola insulin suitable for an ultra-long-acting therapy for diabetes can be manufactured by any recombinant technology, chemically conjugating insulin with protein-binding peptides or RNA technology.

The Magoola insulin configuration also exhibits sustained bioactivity in vivo (e.g., demonstrate glucose lowering activity greater than hours, weeks or months justifying less frequent dosing. The Magoola insulin configurations also demonstrate prolonged system residence time in vivo based on the half-life of the proteins to which it is conjugated, and particularly when it is conjugated with protein-binding peptides that fuse with different endogenous proteins, this way, release or diffusion out of one type of endogenous proteins is protected allowing a much long half-life.

The sustained bioactivity and prolonged residence time of a given Magoola insulin configuration may be predicted by its ability to bind the receptors responsible for the prolonged in vivo elimination of half-lives.

Proposed ultra-long-acting insulin treatments for human clinical use comprise a Magoola insulin making use of human long-life endogenous proteins to prolong their action in vivo. To understand the behavior of Magoola insulin configurations of various designs, Magoola insulin configurations that are useful as ultra-long-acting insulins for dogs are considered first. As a human long half-life endogenous protein, they are not expected to be more immunogenic than any other current means of administering insulin.

The present disclosure relates to a composition of a fusion protein (i.e., an insulin-endogenous protein fusion through a protein-binding peptide) comprising an insulin polypeptide linked via a peptide linker to a protein-binding peptide capable of binding to endogenous proteins.

An insulin polypeptide may be, for example, an insulin or insulin analog produced by (3-cells in the islets of Langerhans within the pancreas. Insulin functions by regulating the absorption of glucose from the blood upon a stimulus, such as increased protein and glucose levels, when insulin is released from 3-cells. It binds to the IR, initiating a signal cascade that affects many mammalian (e.g., human) metabolism aspects.

The amino acid sequence of insulin is strongly conserved throughout evolution, particularly in vertebrates. For example, native canine and porcine insulins differ by only one amino acid from human insulin, native bovine insulin differs by only three amino acids from human insulin, and native feline insulin differs by just four amino acids from human insulin. As used herein, the terms “B-chain or B-chain analog,” “C-peptide,” or “C-chain,” and “A-chain or A-chain analog” refer to the peptide segments of an insulin polypeptide. Insulin is a 51 amino acid hormone containing two peptide chains (i.e., a B-chain and an A-chain) connected via disulfide bonds (e.g., disulfide bonds formed by one or more B-chain cysteine side chain thiols and one or more A-chain cysteine side chain thiols). The A-chain of insulin is 21 amino acids long, and the B-chain is 30 amino acids long. In the native form of insulin, the A-chain contains one intrachain disulfide bond formed by two A-chain cysteine side chain thiols.

1. A Magoola insulin composition comprising a polypeptide representing insulin, proinsulin, or preproinsulin conjugated with a protein-binding peptide or peptides through a flexible linker, wherein the protein-binding peptide is capable of binding to long half-life endogenous proteins.

2. The Magoola insulin composition of claim 1, wherein the long half-life endogenous proteins comprise Elastin, Collagen, IgG1, IgG2, IgG4, Albumin, Transferrin, Factor XIII, IgG3, Pentameric IgM, IgA monomer, and Fibrinogen, or a combination thereof.

3. The Magoola insulin composition of claim 1, wherein the sequence of Magoola vaccine comprises protein-binding peptide-linker-proinsulin or proinsulin-linker-protein-binding peptide.

4. The Magoola insulin composition of claim 1, wherein a linker comprises GGGGS (SEQUENCE NO 2), GGGGAGGGG (SEQUENCE NO 3), GGGGSGGGG (SEQUENCE NO 4), GGGGGAGGGG (SEQUENCE NO 5), GGGGSGGGGSGGGGSGGGG (SEQUENCE NO 6), GGGGKGGGGKGGGGKGGGG (SEQUENCE NO 7), GGGGGAGGGGAGGGGAGGGGG (SEQUENCE NO 8), GGGGGQGGGGQGGGGQGGGGG (SEQUENCE NO 9), SGGGGQGGGGQGGGGQGGGGG (SEQUENCE NO 10), HGGGGQGGGGQGGGGQGGGGG (SEQUENCE NO 11), PGGGGGQGGGGQGGGGQGGGGG (SEQUENCE NO 12), GGGGGQGGGGQGGGGQGGGGGQGGGG (SEQUENCE NO 13), or a combination thereof.

5. The Magoola insulin composition of claim 1, wherein the protein-binding peptide comprises LRHYY (SEQUENCE NO 14), DIE (SEQUENCE NO 15), DIIP (SEQUENCE NO 16), YRELRLQR (SEQUENCE NO 17), YLSPYILSTL (SEQUENCE NO 18), RLIEDICLPRWGCLWEDD (SEQUENCE NO 19; Ac-RLIEDICLPRWGCLWEDD-NH2), HAIYPRH (SEQUENCE NO 20), THRPPMWSPVWP (SEQUENCE NO 21), GPOGPOGPOGPOGPOGPOGPOGPOGPOGPO (SEQUENCE NO 22), TVELQGVVPRGVNL (SEQUENCE NO 23), QFQSIYQY (SEQUENCE NO 24), VQQWRRIR (SEQUENCE NO 25), DLEALFYK (SEQUENCE NO 26), VGVAPG (SEQUENCE NO 27), TRP (SEQUENCE NO 28), RGD (SEQUENCE NO 29), or a combination thereof.

6. The Magoola insulin composition of claim 5, wherein multiple protein-binding peptides capable of binding to each long-acting endogenous proteins are conjugated using a flexible linker.

7. The Magoola insulin composition of claim 6, wherein at least one protein-binding peptide capable of conjugating with at least one endogenous protein are conjugated with a flexible linker.

8. The Magoola insulin composition of claim 1, wherein the Magoola insulin is produced by a recombinant method, by chemically conjugating with insulin, proinsulin, or preproinsulin manufactured by a recombinant process, or RNA technology.

9. The Magoola insulin composition of claim 8, wherein RNA technology comprises linear mRNA, self-replicating mRNA, circular RNA, and self-replicating RNA.

10. The Magoola insulin composition of claim 9, wherein the RNA technology reduces the dose of RNA administered by 1/1000 to 1/1,000,000 in dose basis of RNA vs. insulin dose.

11. The Magoola insulin composition of claim 1, wherein the Magoola insulin is injected weekly, monthly, or yearly.

12. The Magoola insulin composition of claim 1, wherein the Magoola insulin is injected through an insulin pump.

13. The Magoola insulin composition of claim 1, wherein the dosing of the Magoola insulin is adjusted based on patient sensitivity and need for insulin, modulating the dosing or frequency of administration.
